(19) United States
(12) Patent Application Publication
(10) Pub. No.: US 2022/0310240 A1
(43) Pub. Date: Sep. 29, 2022

(54) SYSTEMS AND METHODS FOR HOSPITAL ASSET LOGISTICS OPTIMIZATION

(71) Applicant: KONINKLIJKE PHILIPS N.V., EINDHOVEN (NL)

(72) Inventors: THOMAS ERIK AMTHOR, HAMBURG (DE); MICHAEL PROKLE, BOSTON, MA (US); KARIN KLABUNDE, BOCHUM (DE); RICHARD MOESSEL, HAMBURG (DE)

(21) Appl. No.: 17/615,860

(22) PCT Filed: May 25, 2020
(86) PCT No.: PCT/EP2020/064378

§ 371 Date: Dec. 2, 2021

Related U.S. Application Data
(60) Provisional application No. 62/856,259, filed on Jun. 3, 2019.

(51) Int. Cl. G16H 40/20

(52) U.S. Cl. CPC G16H 40/20

(57) ABSTRACT

A system (10) for hospital asset logistics optimization includes a real-time locating service (RTLS) (18) configured to perform tracking of current locations of hospital personnel and items of medical equipment, wherein the tracking is referenced to a hospital map. At least one electronic processor (16) is programmed to: identify and/or receive identification of items of medical equipment to be transported and destinations for the respective items of medical equipment to be transported; associate the items of medical equipment to be transported with individuals from amongst the hospital personnel based on the current locations of the items of medical equipment to be transported and the current locations of the associated individuals; and transmit transport requests to associated mobile devices (52) of the associated individuals wherein each transport request identifies at least the item of equipment to be transported that is associated with the individual and its destination.

### FIELD

The following relates generally to the medical electronic networking arts, medical item and personnel location arts, medical item logistics arts, medical personnel logistics arts, real time locating service arts, and related arts.

### BACKGROUND

Many mobile medical devices, such as ultrasound, patient monitors, etc., exist in modern hospitals. When such a medical device is needed at one location in the hospital, a nurse, orderly, or other hospital employee is typically tasked with transporting the device from another location in the hospital to where it is needed. In addition, there are many other medical items that need to be transported through the hospital, such as blood samples, paper work, medicine, and tools.

To reduce cost, hospital logistics commonly employ personnel with no medical training, usually referred to as hospital orderlies, for transporting mobile equipment to locations where the equipment is to be used. If a medical item is needed soon or immediately and an orderly is unavailable, then a nurse or other hospital employee may be tasked with retrieving the item. These approaches can be time consuming as the person sent to transport a piece of equipment may need to initially locate the item, then travel some distance to its location, and then transport it to the destination, during which time any medical procedure to be performed using that equipment is delayed.

The following discloses new and improved systems and methods to overcome these problems and others.

### SUMMARY

In one disclosed aspect, a system for hospital asset logistics optimization includes a real-time locating service (RTLS) configured to perform tracking of current locations of hospital personnel and items of medical equipment, wherein the tracking is referenced to a hospital map. At least one electronic processor is programmed to: identify and/or receive identification of items of medical equipment to be transported and destinations for the respective items of medical equipment to be transported; associate the items of medical equipment to be transported with individuals from amongst the hospital personnel based on the current locations of the items of medical equipment to be transported and the current locations of the associated individuals; and transmit transport requests to associated mobile devices of the associated individuals wherein each transport request identifies at least the item of equipment to be transported that is associated with the individual and its destination.

In another disclosed aspect, a non-transitory computer readable medium stores instructions implemented on a device having at least one electronic processor and a display device. The at least one electronic processor is programmed to: upon receiving a transport request, provide a user interface on the display device of the device, the user interface including a plurality of fields including (i) a field to accept the transport request; (ii) a field to reject the transport request; and (iii) a field including a description and a location of an item of medical equipment to be moved; and receive a user input indicative of a selection of one of the field to accept the transport request or the field to reject the transport request.

In another disclosed aspect, a hospital asset logistics optimization method includes: identifying and/or receive, from a plurality of RTLS devices dispersed throughout a medical facility, identification of items of medical equipment to be transported and destinations for the respective items of medical equipment to be transported; generating an instance of a hospital asset logistics optimization workflow using a Business Process Model (BPM) that includes existing work schedules of departments in the medical facility; determining transport paths in a medical facility map for the items of medical equipment to be transported from their respective current locations to their destinations based at least on the generated BPM; predicting paths of the personnel in the medical facility map based at least on the tracking of the personnel and based at least on the generated BPM; associating the items of medical equipment to be transported with individuals from amongst the personnel based on coincidence of the transport paths with the predicted paths of the associated individuals; transmit transport requests to associated mobile devices of the associated individuals wherein each transport request identifies at least the item of equipment to be transported that is associated with the individual and its destination; providing a user interface on a display device of the associated mobile devices, the user interface including a plurality of fields including (i) a field to accept the transport request; (ii) a field to reject the transport request; and (iii) a field including a description and a location of an item of medical equipment to be moved; and receiving a user input indicative of a selection of one of the field to accept the transport request or the field to reject the transport request.

One advantage resides in providing an electronic network operative to reduce personnel hours spent on transport of medical items.

Another advantage resides in providing an electronic system for generating routes for medical personnel to transport an item from one location in a hospital to another.

Another advantage resides in optimizing a path for a hospital staff member to transport an item from one location in the hospital to another based on a current position and a traveling direction of the hospital staff member.

Another advantage resides in minimizing distances, time, and extra movement of items in a hospital by a medical staff personnel.

Another advantage resides in providing an application on user devices of medical personnel staff to accept requests to transport items from one location in a hospital to another hospital location in the hospital.

Another advantage resides in providing user alert devices integrated into personal mobile devices and/or disposed on portable medical items and operating in conjunction with an electronic medical logistics network to facilitate efficient non-redundant transport of medical items.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically shows a hospital asset logistics optimization system according to one aspect.

FIG. 2 shows exemplary flow chart operations of the system of FIG. 1.

FIGS. 3A and 3B show additional components of the hospital asset logistics optimization system of FIG. 1.

FIG. 4 shows an exemplary user interface including information transmitted by the hospital asset logistics optimization system of FIG. 1.

FIG. 5 shows exemplary flow chart operations of the system of FIG. 1.

### DETAILED DESCRIPTION

The following discloses electronic networks operative to reduce personnel hours spent on transport of medical items by combining a logistic application program (“app”) running on cellular telephones, tablet computers, or other mobile devices of hospital staff with a real time locating service (RTLS) that uses RFID technology or the like to track locations of hospital staff and hospital equipment in real time, and optionally further including modeling of hospital workflows, e.g. using a Business Process Model (BPM) in a notation such as BPMN, leveraging existing works schedules of laboratories or other medical departments, or so forth. The model predicts when and where various equipment will be used. A path optimizer uses inputs including current locations of hospital staff and of equipment which needs to be moved, along with knowledge of the destination (all these locations/predicted paths are suitably represented in a hospital floor map or other layout space) to identify the staff person best positioned to pick up and move a piece of equipment to its destination.

The system further includes a logistic application program (“app”), instances of which are running on cellphones or other mobile devices of hospital staff, via which the identified staff person is alerted to the need to move the equipment and provided with its current location and the destination. In a suitable embodiment, the app provides at least “accept” and “decline” buttons via which the staff person responds, and if the staff person accepts the task then the RTLS is used to verify the staff person picks up and moves the equipment (e.g., by detecting coincidence of location and trajectory of the staff person and equipment as measured by the RTLS).

In a variant embodiment, no response via the app is delivered, and rather the RTLS is used to confirm acceptance of the task by detecting coincident movement of the staff person and the item. In addition to identifying the equipment and location, in some embodiments the logistics app may provide the accepting staff person with a turn by turn navigation system using the hospital map and RTLS information to guide the staff person transporting the equipment to the destination.

In some embodiments disclosed herein, instead of (or in addition to) relying upon the model to predict an equipment move request and its destination, a staff person could use the app to affirmatively request that a piece of equipment be moved to a certain destination at a certain time. Rather than identifying a single staff person optimally positioned to transport a piece of equipment, the path optimizer may identify a ranked list of such staff persons, and then query the highest ranked person on the list; if they decline then go to the next-highest ranked person on the list, and so forth.

In other embodiments disclosed herein, the app can include an input dialog via which the person transporting the equipment can indicate if any issues are observed with that equipment (e.g. visible damage, a low battery indicator, or so forth). This can serve as additional input to the model which may, for example, take that piece of equipment out of service and identify available substitute equipment.

In further embodiments disclosed herein, optical notification devices (e.g. lamps) can be installed on various pieces of portable equipment, which are lighted to indicate the equipment needs to be transported. In a further variant, the lamp can have an associated display showing information such as the destination and/or the name of a staff person who accepts the transport request.

In yet other embodiments disclosed herein, staff who accept transport tasks are given some sort of incentive (e.g. monetary, extended break time, or so forth) in proportion to the number of transport tasks accepted, to total transport distance, or other suitable metric(s).

In other embodiments disclosed herein, constraints may be placed on which staff are competent to perform a given transport task. Constraints might, for example, include authorization to enter the destination location (e.g. staff considered for transport of an item to an ICU with restricted access may be limited to those staff with ICU access privileges), physical fitness requirements for transporting a heavy piece of equipment, or so forth. If two or more people are required to move a certain piece of equipment, then the path optimizer may take this into account by identifying and querying two (or more) staff persons. In this case, when the second person accepts both are notified as to who has accepted the transport request, so they recognize each other when they meet at the current location of the piece of equipment. If it is unsafe for a single person to move that piece of equipment, it is further contemplated to issue a warning via the app if the RTLS detects coincidence of location and trajectory of the piece of equipment and only a single staff person. (Conversely, in an incentivized system, if two or more staff persons are moving in coincidence with a piece of equipment that requires only a single person for transport, then the incentive may be split between the two persons).

The systems disclosed herein can include several components: some source of task identification (modeling and/or an equipment request user dialog provided via the app); the RTLS; the path optimizer; and the app programmed to communicate with the RTLS and the path optimizer.

The following also discloses a path optimization algorithm. Unlike a taxi or peer-to-peer ride sharing call request app, in which a ride request is typically broadcast to all drivers within a certain radius of the requestor, the path optimization here performed optimally identifies a single staff person (or, perhaps, a “top N” ranked list of staff persons) for performing the transport task. The optimization preferably also takes into account variables such as: the locations of all staff members currently on duty, as well as the current locations of equipment to be moved and their respective destinations (and possibly also constraints on arrival times). The optimal person to ask to perform a given transport task may not be the person closest to either the current location of the equipment or its destination; rather, the goal is to match a predicted travel path of a staff person with the equipment transport path.

As used herein, the term “BPM” (and variants thereof) refer to a model of a process or workflow in which nodes or blocks represent activities, events, decision points (e.g. gateways), and so forth, and connections indicate flow of the process and/or data into or out of the process. BPMs receive input from data sources (e.g., mined from the various hospital databases in the case of a BPM representing a medical process or workflow) and provide real-time monitoring of the process as modeled by a model. A common BPM formalism is Business Process Model and Notation (BPMN) (e.g. BPMN 2.0, available from Object Management Group, Needham, Mass., USA) which uses a block diagram notation constructed by user employing a graphical user interface. A BPMN representation of a process typically employs elements such as: flow objects representing events which occur or activities to be done; gateways representing the forking or merging of paths; connectors representing process flow, data flow, or linking flow objects; grouping elements such as swim lanes; and/or so forth. The BPM for a given medical process or workflow is typically generated by a user interacting with a graphical user interface (GUI) which enables the user to construct the BPM by adding and configuring elements and connectors. The BPM modeling GUI may be preconfigured for the given hospital or other medical facility, e.g. providing automatic connection of an element to the appropriate medical databases for receiving data to be processed at the element and for outputting data generated at the element to appropriate databases.

While a BPM can provide beneficial fine resolution as to the likely movements of hospital personnel and medical items, less sophisticated path optimization algorithms are also contemplated. For example, the path optimization can rely upon the RTLS and hospital map to predict a most likely path of each person based on the person's current location and trajectory from the RTLS and the available paths forward from that location along that trajectory as defined in the hospital map. Such RTLS and map-based modeling can further incorporate time-dependent population density to provide probabilistic estimates of the paths of staff persons. For example, if it is around noon and the staff person's trajectory passes close to the lunch room which has a high population density around noon, it may be reasonable to predict that the lunch room is the intended destination. Further refinement could be based on individual roles, departmental assignments, or other information—for example, a cardiologist is more likely to be headed to the cardiology department as compared with, e.g., the neonatal ward. Historical information may also be leveraged, e.g. for each person a map could be maintained of the amount of time the person spends in various areas of the hospital, and then the most probable destination is selected as one of these high probability areas (specific to that person; or in another embodiment, specific to persons of a common cohort).

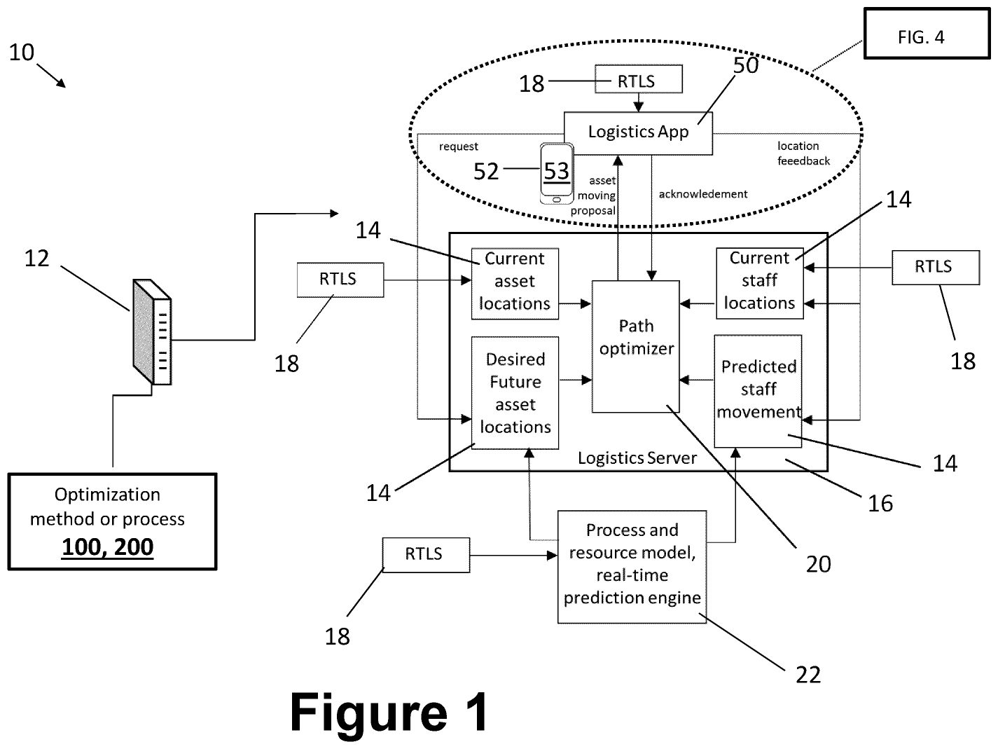

With reference to FIG. 1, an illustrative hospital asset logistics optimization system 10 is shown. As shown in FIG. 1, the system 10 includes a server 12 implementing one or more databases 14 and at least one electronic processor 16, and an RTLS system 18. (Note, FIG. 1 diagrammatically shows the RTLS 18 four times, connected with different components. This is for illustrative convenience—in practice, there is a single RTLS 18 although it may comprise a number of different RFID tag readers, swipe card stations, GPS receivers, and/or other RTLS devices). The at least one electronic processor 16 of the server 12 is configured to use data received from the RTLS devices 18 and, along with information retrieved from the one or more databases 14, generate an optimized route for a medical staff member to transport an item from a first or initial location in a hospital or medical facility to another or final location in the hospital.

The database(s) 14 can be any suitable database, such as a Health-Level 7 (HL7)-compliant database, an Electronic Medical Record (EMR) or similar database such as an Electronic Health Record (EHR), various clerical databases such as a hospital admissions and/or human resources databases, various combinations thereof, and so forth.

The database(s) 14 can be stored on one or more non-transitory storage media which may, by way of non-limiting illustrative example, include one or more of a magnetic disk, RAID, or other magnetic storage medium; a solid state drive, flash drive, electronically erasable read-only memory (EEROM) or other electronic memory; an optical disk or other optical storage; various combinations thereof; or so forth; and may be for example a network storage accessible by the server 12, an internal hard drive of a workstation (not shown), various combinations thereof, or so forth. It is to be understood that any reference to a non-transitory medium or media herein is to be broadly construed as encompassing a single medium or multiple media of the same or different types.

Likewise, the electronic processor 16 may be embodied as a single electronic processor or as two or more electronic processors. The at least one electronic processor 16 is operatively connected with the database(s) 14. The electronic processor 16 can be any suitable processor, typically a single server computer or a plurality of server computers (e.g. interconnected to form a server cluster, cloud computing resource, or so forth), although the electronic processor 16 may be alternatively or additionally embodied as a computing device (e.g., typically a workstation computer, or more generally a computer, although another form factor such as a tablet, a smartphone, and so forth is also contemplated). While a single server 12 is illustrated, it will be appreciated that the desired computing capacity may be obtained by way of a plurality of cooperating server computers, e.g. a computing cluster, cloud computing resource, or so forth, and it is to be understood that the server computer 12 encompasses such multi-computer embodiments.

The RTLS 18 is configured to perform tracking of current locations of hospital personnel and medical equipment in which the tracking is referenced to a hospital map (which, while not shown, can be stored in the database(s) 14). The RTLS 18 generates location data of the hospital staff members or personnel and medical equipment on an occasional basis, and stores this data in the database(s) 14 (or, alternatively, the RTLS devices may be accessed on an as-needed basis to identify the location of a staff person). For example, the RTLS 18 can track the locations of the staff and can transmit this data to the server 12 for storage in a first database, and the RTLS 18 can also track locations of the medical equipment items to be moved can transmit this data to the server for storage in a second database.

In some examples, the RTLS device(s) 18 include a GPS device configured to obtain GPS location data of the staff members or the medical equipment items to be moved. By way of non-limiting illustration, one example of a suitable RTLS device 18 is an RFID-based RTLS employing radio frequency identification (RFID) tags worn by staff e.g., on a wristband, an article of clothing, an identification badge), disposed on or in tracked equipment, or so forth and tracked by RFID tag readers placed at strategic locations around the hospital or other medical facility to allow for remote location monitoring of the patient or staff member. An electronic map of the hospital or other medical facility stored in the database(s) 14 identifies the location based on which RFID tag reader picks up the RFID tag (or, in a more advanced embodiment, detection of the RFID tag by two or three RFID tag readers enables more precise location by way of triangulation).

In another non-limiting illustration, the RTLS 18 can employ a smartphone, a tablet, or another smart device operated by the staff member. In this example, the user can log-in into a mobile application (“app”) on their smartphone or tablet and use the global positioning system (GPS) in the phone or tablet to collect position information and determine a location of the staff member or patient. The RTLS 18 optionally further leverages other locational information such as swipe events recorded by a swipe card reader controlling access to a restricted area. The RTLS 18 may utilize multiple tracking modalities, e.g. RFID and GPS for example, and average the results, select the most reliable result, or otherwise combine tracking from the different tracking modalities. For example, GPS can be quite accurate for tracking a person carrying a GPS-equipped mobile device (e.g. cellphone) outside, e.g. passing between buildings of a hospital campus and possibly out of range of any RFID tag readers; on the other hand, when the person enters a building the interior location may degrade or completely block the GPS satellite signals at which point the RTLS 18 suitably switches to RFID-based tracking using RFID tag readers locates inside the building. The server 12 at the medical facility can then use the determined location from the RTLS 18 and generate a route for the staff member to show where an item is to be moved, which can be displayed on the smartphone or tablet.

The system 10 is configured to perform a hospital asset logistics optimization method or process 100. The database(s) 14 stores instructions which are readable and executable by the at least one electronic processor 16 and to perform disclosed operations including performing the hospital asset logistics optimization method or process 100. In some examples, the method 100 may be performed at least in part by cloud processing.

The instructions which are executed to perform the hospital asset logistics optimization method or process 100 may be viewed as implementing a path optimizer module 20; and, in some embodiments, a process and resource model real-time prediction engine 22.

The path optimizer module 20 is configured to (i) retrieve the location data collected by the RTLS 18 for both the staff personnel and medical items; and (ii) upon receiving a request from a medical staff member for a medical item to be transferred to the location of the medical staff member, generate an optimized path for an optimally selected staff personnel member to transport the desired medical item from its current location to the location requested by the medical staff member. In some embodiments, the path optimizer module 20 can generate the pathway using a BPM generated by the process and resource model real-time prediction engine 22.

In a practical implementation, there can be many process models for monitoring a corresponding number of many different medical processes or workflows; a single illustrative process model is discussed here for simplicity, and is assumed to be implemented as a BPM which employs BPMN notation in illustrative examples herein. Such process models can include complex elements like branching, decisions, loops, parallel paths. In one example embodiment, the path optimizer module 20 and the process and resource model real-time prediction engine 22 are both implemented in the server 12, while in other embodiments, the path optimizer module and the process and resource model real-time prediction engine are implemented in separate servers.

The path optimizer module 20 is configured to calculate the optimum paths for staff to move through the hospital in order to reach their desired future locations and at the same time pick up and take assets with them, so that those assets will be located at the correct positions where they will be needed next. If the location of both assets and staff can be tracked via the RTLS device 18 (e.g. because the staff wear badges with RTLS-compatible RFID tags and the various medical items are similarly tagged with RTLS-compatible RFID tags), then it can also be detected automatically that they started moving together. The staff and assets involved in such a transport procedure do not necessarily need to be connected to the same process. For example, a nurse N1 moving from department A to department B to visit a patient as part of a clinical process P1 may be requested by the logistics app to pick up an ultrasound device on the way to bring it to department B where it is expected to be required 10 minutes later for a different clinical process P1. In this way, nurse N2 at department B does not need to fetch the ultrasound device, which would have meant walking to a different location and back. It is also possible to ‘drop’ the asset in some other department C that is passed on the route to department B.

By way of non-limiting illustrative example, the path optimizer module 20 can, for example use the following algorithm to generate the optimum paths. The algorithm is defined as pᵢ (x_{i,0}, x_{i,1}, . . . , x_{i,n}, a_{i,1}, . . . , a_{i,n}) which represents a total path of person i currently located at location x_{i,0} with expected future locations x_{i,1} . . . x_{i,n} throughout the currently foreseeable future processes carrying assets a_{i,j} on the path intervals x_{i,j-1}→x_{i,j}, respectively, where a_{i,j} is the unique number of an asset or zero if no asset is carried. A simple implementation of the path optimizer module 20 would search a solution for all a_{i,j} to minimize the total path length of all persons Σᵢ pᵢ. The minimization should be subject to the correct locations of all assets at the time they are needed, and no asset can be transported by two or more persons during overlapping time intervals. A regularization term, such as λΣᵢa_{i,j}∥₀, penalizing multiple asset transports by a single person can be added to the objective function to distribute the work more equally among staff.

As shown in FIG. 1, the system 10 further includes an application program (“app”) 50 (also referred to herein as a logistics app 50) which is loaded on, and executable on, mobile devices 52 of the medical staff personnel (e.g., an illustrative cellular telephone 52, or a tablet computer, personal data assistant or PDA, and/or so forth). The app 50 may be downloaded to the mobile device 52 from an app store accessed via a Wi-Fi, cellular, or other wireless communication network, or may be loaded onto the mobile device 52 by hospital information technology (IT) personnel before the mobile device 52 is assigned to a hospital staff person. In a suitable embodiment, the app 50 is represented on the home screen or applications screen of the mobile device 52 as an app icon (i.e. a small square, round, or other compact graphical element representing the app 50) and the user launches (i.e. initiates running of) an instance of the app 50 on the device 52 by touching the icon on the (touch-sensitive) screen 53 of the mobile device 52.

The app 50 can provide various functionality. It can provide the user interface (UI) via which the logistics server 16 sends a transport request to a specific staff person. It can receive an assent from the staff person to fulfill the transport request, or alternatively receive the staff person's selection to decline the transport request. In some embodiments, once a transport request has been accepted, the app 50 may leverage the RTLS 18 and the hospital map to provide turn-by-turn directions for the staff person to reach the current location of the device and then to guide the staff person in transporting the device to its destination.

In some embodiments, the app 50 may also be used to manually initiate a transport request. For example, a clinician may enter a request for an echocardiogram device (for example) to be delivered to a specific patient's room. The app can provide various dialog formulations for initiating such a request, such as a series of drop-down selection lists such as: “Medical device”→“Cardiology”→“Echocardiogram” to select the device and a location drop-down list enabling drilling down to select a particular location or to select the clinician's current location (a useful option since the clinician may often be at the location to which he or she wishes to have the equipment delivered.)

The app 50 may provide other functionality. In some embodiments, a current location of a staff member may be provided manually by the staff member via the app 50. This can be useful, for example, if the staff person is located somewhere that is out-of-range of the RTLS 18. In this latter case, the users of the app 50 can select their own location, for example by clicking on a map of the hospital, to provide this information to the server 12. Furthermore, the future location of staff can also be provided manually by the staff member via the logistics app 50.

In one embodiment, the resource model real-time prediction engine 22 contains data of currently-ongoing hospital processes, for example clinical pathways of patients or radiology processes. This may be done using a BPM in some embodiments. In other embodiments, trajectory projections (optionally probabilistic) are made for personnel based on current location/trajectory and the hospital map. The path optimizer module 20 uses this information to estimate the probability of the (i.e., desired) future location of staff and assets at any future time. This is used to pre-allocate equipment to minimize interruption and wait times due to unavailable assets and equipment.

In some embodiments, the resource model real-time prediction engine 22 is configured to prioritize the tasks computed by the path optimizer module 20. For example, the users of the app 50 can indicate a priority level (e.g., emergency, urgent, not urgent, and so forth) of the particular task. In addition to using the current path and trajectory of the medical personnel, the path optimizer module 20 can use the priority level to rank the list of tasks.

In another contemplated variant, the resource model real-time prediction engine 22 is configured to schedule the tasks based on a next task, or a future task. For example, while it might be most efficient for employee A to complete task X based on location, there may be a corresponding task Y that would need to be completed after task X, wherein the efficiency of tasks X and Y actually dictate employee B is better suited. This can be implemented in the above-described example of the path optimizer module 20 by introducing time-based constraints, e.g. requiring that task Y be performed after task X.

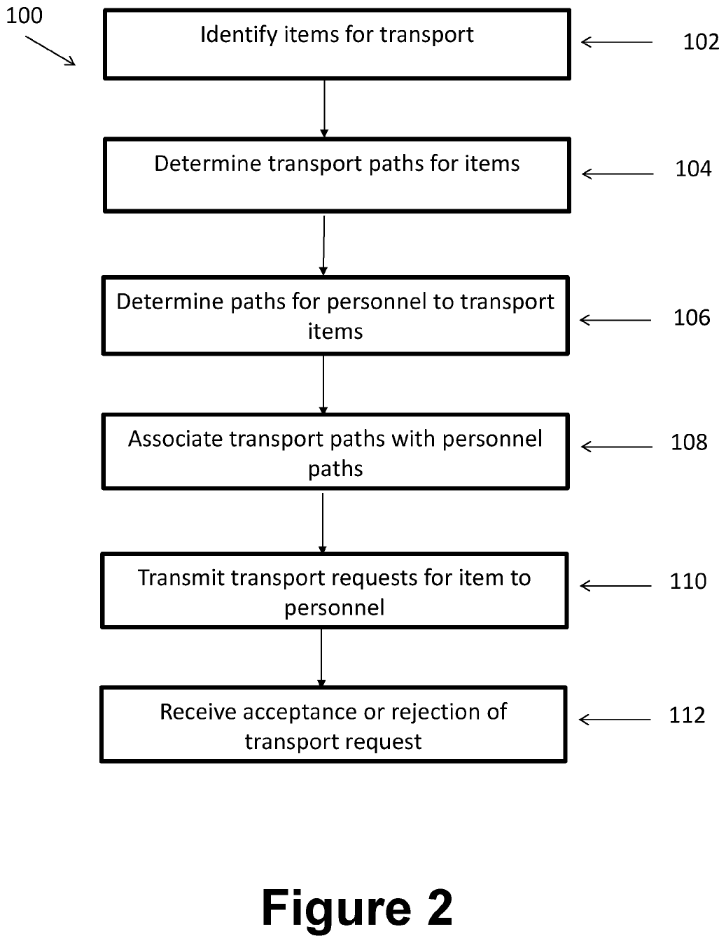

With reference to FIG. 2, an illustrative embodiment of an instance of the hospital asset logistics optimization method 100 executable by the at least one electronic processor 16 is diagrammatically shown as a flowchart. At 102, an identification of items of medical to be transported, along with destinations for the respective items of medical equipment to be transported, are identified or received. This identification can be a manual operation, for example a user operating an instance of the app 50 to request that a particular medical item be delivered to a particular location. Additionally or alternatively, this identification can be automated, for example by determining a probable location where the medical item will next be needed based on the prediction engine 22. Optionally, a time at which the item will be needed at the destination is also identified at 102. If no specific time is identified, then the time may be set to a value indicating “as soon as possible”. In some embodiments, a transport operation may be designated as “high priority” or as some other expedited indication.

At 104, transport paths are determined in the hospital map for the items of medical equipment to be transported from their respective current locations to their destinations. The transport paths only include data collected by the RTLS 18 associated with the items to be transported, and not data from the RTLS 18 associated with the medical staff personnel.

At 106, paths of the hospital personnel are predicted in the hospital map based at least one tracking of the hospital personnel throughout the hospital. These paths only include data collected by the RTLS 18 associated with the medical staff personnel, and not data from the RTLS 18 associated with the items to be transported.

At 108, the items of medical equipment to be transported (e.g. the paths generated at 104) are associated with individuals from amongst the hospital personnel (e.g., the paths generated at 106) based on a coincidence of the transport paths with the predicted paths of the associated individuals. In one embodiment, only the data collected by the RTLS 18 for both the items to be transported and the medical staff personnel is used for this association operation.

In another embodiment, the at least one electronic processor 16 is programmed to generate an instance of a hospital asset logistics optimization workflow using a BPM that includes existing work schedules of departments in the hospital. The paths of the hospital personnel in the hospital map are predicted further based on the generated BPM model. In another example, the BPM is generated further using constraints including (i) constraints related to the hospital personnel in which the constraints include at least an access permission level of each medical personnel; and (ii) constraints related to the items of medical equipment to be moved in which the constraints include at least a size and weight of the item to be moved. Using the generated BPM, the items of medical equipment to be transported and/or the destination where the items should be transported can be automatically identified.

In some embodiments, the associating operation at 108 includes (i) generating a ranked list of candidate individuals for each item of medical equipment to be transported and (ii) selecting the top-ranked candidate individual as the individual associated with the item of medical equipment to be transported.

At 110, one or more transport requests are transmitted to respective instances of the app 50 running on mobile devices 52 of the associated individuals. Each transport request identifies at least the item of equipment to be transported that is associated with the individual and its destination. The individuals associated with the mobile device 52 can either accept or decline the transport requests via the app 50. More nuanced responses are also contemplated, for example acceptance with a specified time delay (e.g. “accept to pick up item within 10 minutes”).

At 112, the server 12 is configured to receive acceptances and/or rejections of the transport requests from the associated individuals via the mobile devices 52 of the associated individuals. For the received acceptances, the server 12 is configured to use the RTLS devices 18 to confirm transport of the corresponding items of medical equipment to be transported to their respective destinations. These confirmations can be conveyed to the individual who accepted the transport request via the app 50.

For any received rejections, the server 12 is configured to associate the corresponding items of medical equipment to be transported with different individuals from amongst the hospital personnel and transmit new transport requests to the mobile devices 52 of the different individuals. To do so, the transport requests are only sent to the top ranked candidate individual from the ranked list. If the top ranked candidate individual rejects the transport request, the server 12 transmits the request to the next-highest ranked individual, and so forth until one of the individuals accepts the request.

In some embodiments, the individual who accepts the transport request can provide feedback to the server 12 regarding a state of the item to be transported. The server 12 is configured to receive, via the mobile device 52 of an associated individual, a notification of a faulty condition of the item of medical equipment to be transported that is associated with that individual. For example, the individual who accepts the request can perform a (i.e., visual) check of the equipment. If there is any visible damage, or if the item seems to be operational, the individual can provide an appropriate notification to the server 12 via the app 50.

The app 50 could then provide a button or selection box to specify if this piece of equipment seems to be operational. In this way, the server could perform a remedial action, e.g., rescheduling the equipment to be moved to exclude potentially broken devices, to automatically inform a service technician, identifying a replacement item to replace the faulty item of medical equipment to be transported; and transmitting an updated transport request with the replacement item.

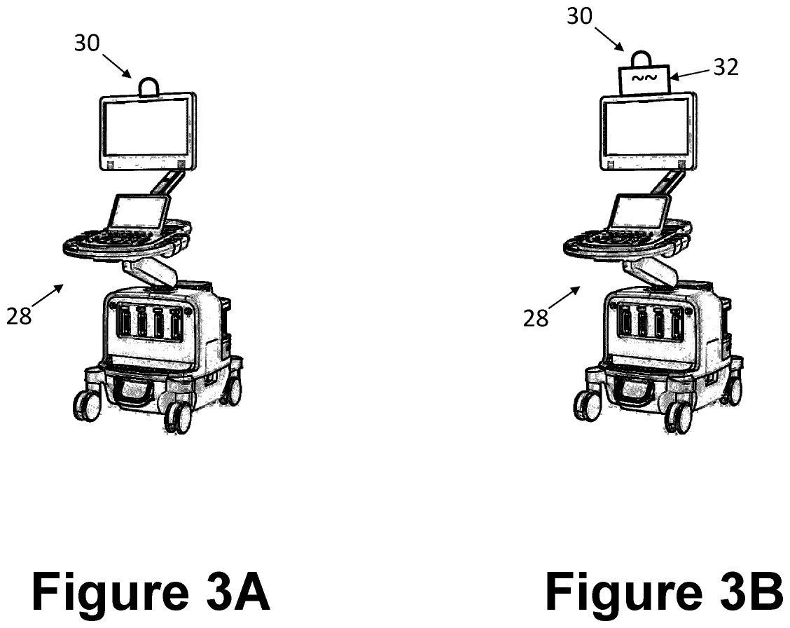

With reference to FIGS. 3A and 3B, the system 10 further includes notification devices 30, 32 disposed on respective items of portable medical equipment (such as an illustrative portable ultrasound machine 28 in illustrative FIGS. 3A and 3B). The at least one electronic processor 16 of the server 12 is programmed to transmit indications to the notification devices 30, 32 of the items of medical equipment to be transported, the indications that the items to the respective associated individuals. In one example, as shown in FIG. 3A, the notification device is a lamp 30 attached to a respective item of medical equipment. The transmitted indications from the server 12 are operative to activate the lamp 30 when the indication is transmitted thereto. In another example, as shown in FIG. 3B, the notification device includes both the lamp 30 and a display device 32 attached to a respective item of medical equipment. The transmitted indications from the server 12 are operative to control the lamp 30 as just described, and to also control the display device 32 to display relevant information about the transport request as a textual or pictorial message when the indication is transmitted thereto. For example, the display device 32 may display the name of the staff person who accepts the transport task to transport the ultrasound machine 28, and/or may display the destination to which the ultrasound machine 28 is to be transported.

In some examples, the medical item to be moved may be required by more than one associated medical staff personnel to move (e.g., a large, long, or heavy piece of equipment). To remedy this problem, the at least one electronic processor 16 is programmed to label the items of medical equipment to be transported as to a number of persons required to perform the transport. For items labelled to be transported by more than one person, the associate operation at 108 associates the labelled number of individuals with the item. If the medical item requires two people to transport, but the RTLS 18 detects only a single person coincidently moving with the medical item (thus indicating that only a single person is transporting the medical item) then a warning may be issued via the instance of the app 50 on the mobile device 52 carried by the (single) person moving the medical item for example, issuing a beeping alert and/or a vibrational haptic alert via the phone vibrator. If the item being moved includes a lamp 30 such as described with reference to FIGS. 3A and/or 3B, then the warning could additionally or alternatively include flashing the lamp 30.

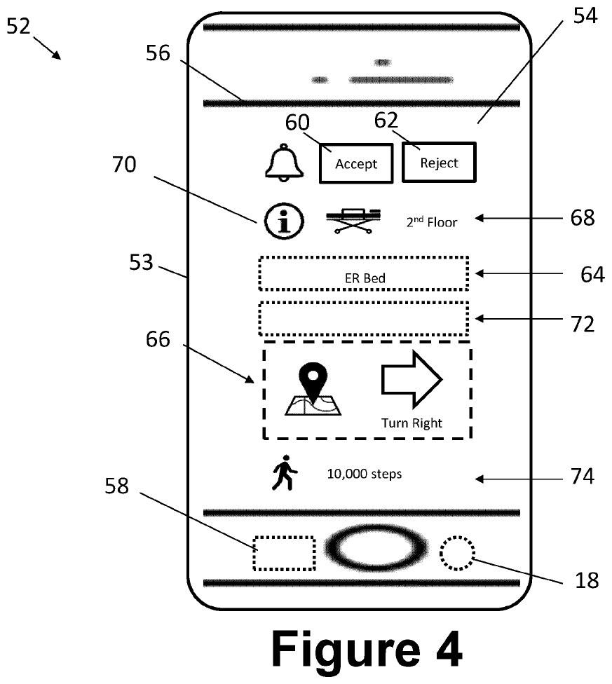

FIG. 4 shows an example of a display suitably shown by an instance of the app 50 running on a user mobile device 52 of an associated hospital staff personnel. The display is suitably shown in the touch-sensitive screen 53 of the mobile device 52 (see FIG. 1). The app 50 provides a user interface 54 for display on a display device 56 of the mobile device 52. The mobile device 52 also includes at least one electronic processor 58, and optionally further includes a GPS sensor communicating with the RTLS 18. Upon receiving the transport request from the server 12, the electronic processor 58 is programmed to provide the user interface 54 on the display device 56. As shown in FIG. 4, the user interface 54 includes a plurality of fields, including (among others): (i) a field 60 to accept the transport request; (ii) a field 62 to reject the transport request; and (iii) a field 64 including a description and a location of a hospital asset to be moved.

Once the user interface 54 is displayed, the electronic processor 58 is programmed to receive a user input (e.g., a finger tap on the display device 56) from the individual of a selection of either the field 60 to accept the transport request or the field 62 to reject the transport request. If the reject field 62 is selected, the electronic processor 58 is programmed to remove the user interface 54 from the display device 54.

If the accept field 60 is selected, the electronic processor 58 is programmed to display additional fields in the user interface 56. As shown in FIG. 4, these additional fields can include: (iv) a field 66 with navigational directions to deliver the item to be moved from a current location to a final location in the hospital; (v) a field 68 including textual or pictorial information related to the item to be moved; (vi) a field 70 including an indication indicative of an urgency level related to the item to be moved; (vii) a field 72 including an communication window enabling textual or pictorial communication between the individual operating the user device 52 and a staff member who requested the item to be moved; and (viii) a field 74 including incentive information for the user for moving the item from a current location to a final location in the hospital.

The user interface 54 can include information from the transport request in one or more of the corresponding fields. For example, navigational directions including step-by-step directions and turn directions from the current location to the final location using the map of a hospital can be displayed in the field 66. In another example, when the item to be moved requires more than one staff member for transport, information related to the “other” staff member(s) can be displayed in the field 72 for the staff members to be able to locate and communicate with each other. In a further example, the incentive information displayed in the field 74 includes one or more of a number of accepted transport requests and a total transport distance of the transport request.

In another embodiment, the transport request can include a desired time at which the item to be moved. For example, the transport request can be sent at 1:00 pm to the user devices 52 and indicate that the item to be moved should be delivered to the final location at 2:00 μm.

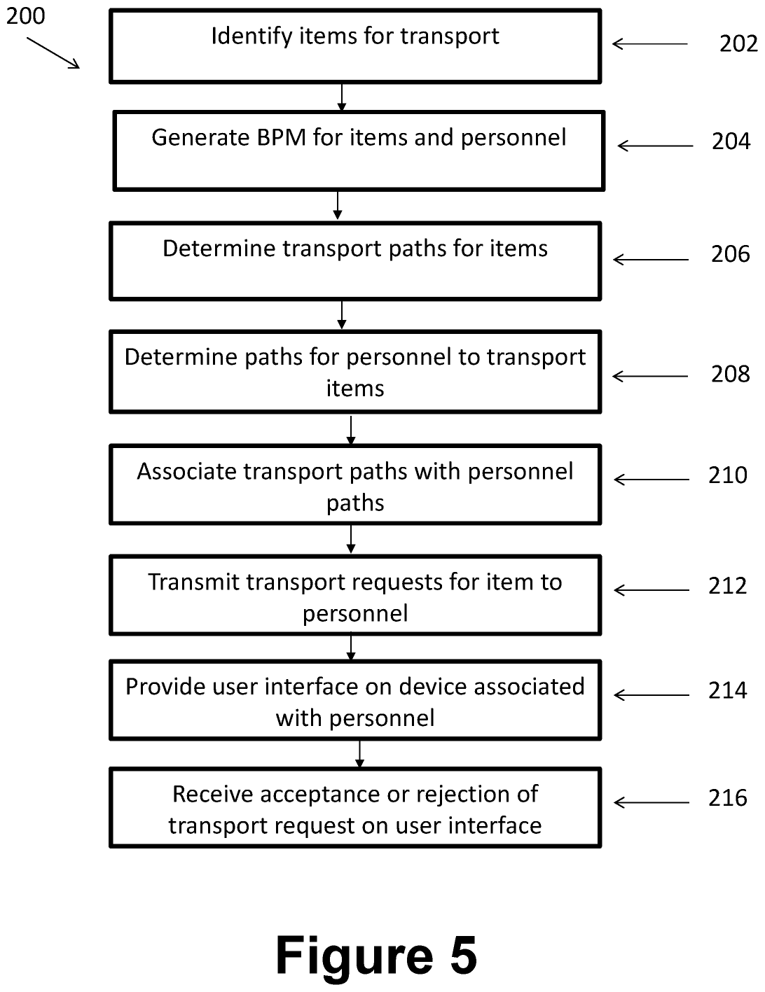

FIG. 5 shows another illustrative embodiment of an instance of the hospital asset logistics optimization method 200 executable by the at least one electronic processor 16 of the server 12 and the at least one electronic processor 58 of the mobile device 52 is diagrammatically shown as a flowchart. At 202, an identification of items of medical equipment to be transported and destinations for the respective items of medical equipment to be transported are identified and/or received from a plurality of RTLS devices 18 dispersed throughout a medical facility. At 204, an instance of a hospital asset logistics optimization workflow is generated using a Business Process Model (BPM) that includes existing work schedules of departments in the medical facility. At 206, transport paths in a medical facility map are determined for the items of medical equipment to be transported from their respective current locations to their destinations based at least on the generated BPM. At 208, paths of the personnel in the map are predicted based at least on the tracking of the hospital personnel and based at least on the generated BPM. At 210, the items of medical equipment to be transported are associated with individuals from amongst the personnel based on coincidence of the transport paths with the predicted paths of the associated individuals. At 212, transport requests are transmitted to associated mobile devices 52 of the associated individuals in which each transport request identifies at least the item of equipment to be transported that is associated with the individual and its destination. At 214, a user interface 54 are provided on a display device 56 of the associated mobile devices 52 in which the user interface includes a plurality of fields including (i) a field 60 to accept the transport request; (ii) a field 62 to reject the transport request; and (iii) a field 64 including a description and a location of an item of medical equipment to be moved. At 216, a user input indicative of a selection of one of the field to accept the transport request or the field to reject the transport request is received.

The disclosure has been described with reference to the preferred embodiments. Modifications and alterations may occur to others upon reading and understanding the preceding detailed description. It is intended that the disclosure be construed as including all such modifications and alterations insofar as they come within the scope of the appended claims or the equivalents thereof.

1. A system for hospital asset logistics optimization, the system comprising:
a real-time locating service (RTLS) configured to perform tracking of current locations of hospital personnel and items of medical equipment, wherein the tracking is referenced to a hospital map; and
at least one electronic processor programmed to:
identify and/or receive identification of items of medical equipment to be transported and destinations for the respective items of medical equipment to be transported;
associate the items of medical equipment to be transported with individuals from amongst the hospital personnel based on the current locations of the items of medical equipment to be transported and the current locations of the associated individuals; and
transmit transport requests to associated mobile devices of the associated individuals wherein each transport request identifies at least the item of equipment to be transported that is associated with the individual and its destination.

2. The system of claim 1, wherein the at least one electronic processor is programmed to associate the items of medical equipment to be transported with individuals by operations including:
determining transport paths in the hospital map for the items of medical equipment to be transported from their respective current locations to their destinations;
predicting paths of the hospital personnel in the hospital map based at least on the tracking of the hospital personnel; and
associating the items of medical equipment to be transported with individuals from amongst the hospital personnel based on coincidence of the transport paths with the predicted paths of the associated individuals.

3. The system of either one of claim 1, wherein the at least one electronic processor is further programmed to:
receive acceptances and rejections of the transport requests from the associated individuals via the associated mobile devices of the associated individuals; and
for the rejections, associate the corresponding items of medical equipment to be transported with different individuals from amongst the hospital personnel and transmit new transport requests to the associated mobile devices of the different individuals; and
for the acceptances, use the RTLS to confirm transport of the corresponding items of medical equipment to be transported to their respective destinations.

4. (canceled)

5. The system of claim 3, wherein:
the associate operation generates a ranked list of candidate individuals for each item of medical equipment to be transported and selects the top-ranked candidate individual as the individual associated with the item of medical equipment to be transported; and
for the rejections, the different individuals are the next-highest ranked candidate individuals from the respective ranked lists.

6. The system of claim 1, wherein the at least one electronic processor is further programmed to:
prioritize the transport tasks based on one or more of a priority level associated with respective transport tasks or one or more future transport tasks.

7. (canceled)

8. The system of claim 2, wherein the at least one electronic processor is further programmed to:
generate an instance of a hospital asset logistics optimization workflow using a Business Process Model (BPM) that includes existing work schedules of departments in the hospital; and
wherein the paths of the hospital personnel in the hospital map are predicted further based on the BPM model.

9. The system of claim 8, wherein the at least one electronic processor is further programmed to:
generate the BPM further using constraints related to the hospital personnel, the constraints including at least an access permission level of each medical personnel.

10. The system of claim 8, wherein the at least one electronic processor is further programmed to:
generate the BPM further using constraints related to the items of medical equipment to be moved, the constraints including at least a size and weight of the item to be moved.

11. The system of claim 8, wherein at least one of the items of medical equipment to be transported and its destination are identified automatically using the BPM.

12. The system of claim 1, wherein the at least one electronic processor is further programmed to:
receive, via the associated mobile device of an associated individual, a notification of a faulty condition of the item of medical equipment to be transported that is associated with that individual; and
perform a remedial action to remediate the faulty condition, the remedial action comprising:
identifying a replacement item to replace the faulty item of medical equipment to be transported; and
transmit an updated transport request with the replacement item.

13. (canceled)

14. The system of claim 1, further comprising:
one or more notification devices disposed on respective items of medical equipment;
wherein the at least one electronic processor is further programmed to:
transmit indications to the one or more notification devices of the items of medical equipment to be transported, the indications identifying the items to the respective associated individuals.

15. The system of claim 14, wherein the one or more notification devices include one or more lamps (30), and the at transmitted indications are operative to:
activate the one or more lamps when the indication is transmitted thereto.

16. The system of claim 14, wherein the one or more notification devices include one or more display devices, and the transmitted indications are operative to:
control the one or more display devices to display the indication as a textual or pictorial message when the indication is transmitted thereto.

17. The system of claim 1, wherein the at least one electronic processor is further programmed to:
label the items of medical equipment to be transported as to a number of persons required to perform the transport; and
wherein, for items labeled to be transported by more than one person, the associate operation associates the labeled number of individuals with the item.

18. A non-transitory computer readable medium storing instructions implemented on a device having at least one electronic processor and a display device, wherein the at least one electronic processor is programmed to:
upon receiving a transport request, provide a user interface on the display device of the device, the user interface including a plurality of fields including (i) a field to accept the transport request; (ii) a field to reject the transport request; and (iii) a field including a description and a location of an item of medical equipment to be moved; and
receive a user input indicative of a selection of one of the field to accept the transport request or the field to reject the transport request.

19. The non-transitory computer readable medium of claim 18, wherein the at least one electronic processor is programmed to, upon receiving the user input, one of:
(i) when the user input is indicative of a selection of the field to reject the transport request, remove the user interface from the display device; and
(ii) when the user input is indicative of a selection of the field to accept the transport request, display at least one additional field on the user interface including a field with navigational directions to deliver the item to be moved from a current location to a final location.

20. The non-transitory computer readable medium of claim 19, wherein the at least one electronic processor is further programmed to:
providing, in the field with navigational directions of the user interface, step-by-step directions and turn directions from the current location to the final location using a map of a hospital.

21. The non-transitory computer readable medium of claim 18, wherein the user interface further includes at least one of:
a field including textual or pictorial information related to the item to be moved;
a field including an indication indicative of an urgency level related to the item to be moved;
a field including an communication window enabling textual or pictorial communication between a user of the device and a staff member who requested the item to be moved; and
a field including incentive information for the user for moving the item to be moved from a current location to a final location, the incentive information including one or more of a number of accepted transport requests and a total transport distance of the transport request;
wherein the at least one electronic processor is further programmed to provide, in the field, and indication that the item to be moved it to be moved at an indicated future time.

22. The non-transitory computer readable medium of claim 21, wherein, when the item to be moved requires more than one user, the at least one electronic processor is further programmed to:
display, in the field information related to other individuals who accepted the transport request to transport the item to be moved.

23. (canceled)

24. A hospital asset logistics optimization method, comprising:
identifying and/or receiving, from a plurality of real-time locating service (RTLS) devices dispersed throughout a medical facility, identification of items of medical equipment to be transported and destinations for the respective items of medical equipment to be transported;
generating an instance of a hospital asset logistics optimization workflow using a Business Process Model (BPM) that includes existing work schedules of departments in the medical facility;
determining transport paths in a medical facility map for the items of medical equipment to be transported from their respective current locations to their destinations based at least on the generated BPM;
predicting paths of the personnel in the medical facility map based at least on the tracking of the personnel and based at least on the generated BPM;
associating the items of medical equipment to be transported with individuals from amongst the personnel based on coincidence of the transport paths with the predicted paths of the associated individuals;
transmit transport requests to associated mobile devices (52) of the associated individuals wherein each transport request identifies at least the item of equipment to be transported that is associated with the individual and its destination;
providing a user interface on a display device of the associated mobile devices, the user interface including a plurality of fields including (i) a field to accept the transport request; (ii) a field to reject the transport request; and (iii) a field including a description and a location of an item of medical equipment to be moved; and
receiving a user input indicative of a selection of one of the field to accept the transport request or the field to reject the transport request.
